(19) United States
(12) Patent Application Publication
(10) Pub. No.: US 2015/0025500 A1
(43) Pub. Date: Jan. 22, 2015

(54) APPARATUS AND METHOD FOR INTRAOSSEOUS FLUID INFUSION

(71) Applicants: North Carolina State University, Raleigh, NC (US); 410 Medical Innovations, LLC, Chapel Hill, NC (US)

(72) Inventors: Mark Piehl, Chapel Hill, NC (US); Elizabeth Moody Davenport, Mooresville, NC (US); Alex Eller, Lansing, NC (US); Ashley Hayes, Winston-Salem, NC (US); Laura Johnson, Raleigh, NC (US); Denise J. Witman, Raleigh, NC (US)

(73) Assignees: North Carolina State University, Raleigh, NC (US); 410 Medical Innovations, LLC, Chapel Hill, NC (US)

(21) Appl. No.: 14/214,977

(22) Filed: Mar. 16, 2014

Related U.S. Application Data
(60) Provisional application No. 61/800,400, filed on Mar. 15, 2013.

(51) Int. Cl. A61B 17/34; A61M 39/24; A61M 5/315

(52) U.S. Cl. CPC A61B 17/3472; A61B 17/3498; A61M 5/31581; A61M 39/24
USPC 604/506; 604/223

(57) ABSTRACT

A system allows for rapid intraosseous delivery of fluids from a fluid reservoir via an intraosseous port having a tip disposed within the bone marrow of a patient. The system includes a housing with a chamber and a plunger. An actuator is moveable between first and second positions to move the plunger between the distal and proximal positions. A valve housing includes a first one-way valve fluidly coupled to the fluid reservoir, and a second one-way valve fluidly coupled to the intraosseous port. The valves of the valve housing are oriented such that movement of the actuator from the first to the second position moves the plunger from the distal to the proximal position, drawing fluid from the fluid reservoir through the first valve and into the chamber. Movement of the actuator from the second to the first position moves the plunger from the proximal to the distal position, driving fluid from the chamber through the port and second valve and through the intraosseous port. A grip manipulated by a user effects movement of the actuator, which provides sufficient mechanical advantage for the user to easily overcome resistance to the flow of fluid into the bone marrow. The plunger may be biased in the proximal position such that it is automatically retracted to that position, causing re-filling of the chamber, when the user releases forces imparted to the grip. The tubing and valve mechanism are of sufficient caliber to allow rapid and low-resistance flow of fluid from the reservoir into the chamber. The combination of these components allows rapid infusion of large volumes into the bone marrow without user fatigue, and requiring only one hand to operate the mechanism.

This application claims the benefit of U.S. Provisional Application No. 61/800,400, filed Mar. 15, 2013, which is incorporated herein by reference.

### BACKGROUND

Rapid fluid administration is essential for patients suffering from a variety of life-threatening illness including septic shock, trauma with significant blood loss, severe dehydration, and anaphylaxis. The American Heart Association's PAL's (Pediatric Advanced Life Support) Guidelines recommend fluid resuscitation volumes of 20 ml per kilogram of body weight over a period of 5 minutes. Intravenous (IV) and intraosseous (IO) fluid administration are the two primary methods of rapid fluid resuscitation in these emergent situations. In certain patients dehydration, low blood pressure or other factors can make it difficult to establish venous access for IV fluid administration. This is particularly true in children. In such patients, the IO route is preferred.

IO access is achieved by inserting a specialized needle into one of the long bones of the leg or arm. A fluid source is connected to the IO port, allowing fluids to be delivered into the bone marrow and to thus flow directly into the systemic circulation. Typical entry sites for the IO port include the anterior tibia, the distal femur, the humeral head , and in adults the manubrium of the sternum. Commercially available systems for placing IO needles include the Bone Injection Gun (B.I.G., Waismed, Houston, Tex.), the EZ-IO (VidaCare Corp., San Antonio, Tex.), and the FAST1 adult intraosseous infusion system (Pyng Medical Corp., Richmond, British Columbia, Canada).

The standard set of components used to deliver fluids through an IO port includes a fluid reservoir, a syringe, a three-way stopcock, and IV tubing linking these components with the IO port. The user withdraws the plunger to fill the syringe from the fluid reservoir, turns the stopcock, and then depresses the plunger to drive the fluid through the IO port and into the bone marrow. The process is repeated multiple times until the desired volume has been delivered. Alternatively, one provider fills syringes from the IV fluid bag, while another connects the syringe, administers the fluid, disconnects the empty syringe, and repeats the process.

It is well documented that resistance to fluid flow in the bone marrow is high, often requiring the user to generate pressures of 300-450 mmhg to achieve adequate flow rates. High resistance to fluid flow into the bone marrow represents a key barrier to the rapid administration of fluid via the IO route. The increased resistance requires emergency healthcare providers to either: 1) use great force with a large-volume syringe, often with two hands, and quickly resulting in user fatigue, or 2) to refill a small-bore syringe multiple times to achieve adequate volume, resulting in slow administration times and significant distraction for one or more workers. In either case two providers are often necessary, with one user infusing the fluid, and the other refilling syringes or operating the stopcock.

Medical providers are best able to deliver these pressures manually using a small bore (10 or 20 ml) syringe to overcome bone marrow resistance. However, significant manual force, and repetitive filling and refilling of the syringe are therefore needed to achieve appropriate flow rates. Using the conventional set-up, users quickly become fatigued, and are consumed with the work of filling and refilling, distracting one (or two) providers from tending to other necessary assessment and care, and possibly limiting the effectiveness of resuscitation if the fluid is not delivered rapidly enough.

Consider the example of a 40 kg child with traumatic injury and massive blood loss. This child may require rapid infusion of 40-80 ml/kg of blood products, for a total of 1600-3200 ml.

Repeated doses using a standard technique and 20 ml syringe would require 80-160 injections and the full attention of two healthcare workers, resulting in slow resuscitation and inefficient use of resources. The total infusion time could be 15-20 minutes, well outside the range of recommended rates, particularly in an actively bleeding child.

The present application describes a system that overcomes these limitations, allowing more rapid and efficient IO fluid delivery than could be achieved using existing equipment.

### BRIEF DESCRIPTION OF THE DRAWINGS

FIG. 1 is a side elevation view of a fluid injection system.

### DETAILED DESCRIPTION

Disclosed herein is a system which provides higher infusion pressure that prior art systems with less hand fatigue, and which rapidly refills to allow rapid fluid resuscitation. The system allows for flow rates of at least 50 ml/min or higher (e.g. between 50-200 ml/min) into the bone marrow—rates which are not achievable using the prior art IO fluid delivery systems.

### System

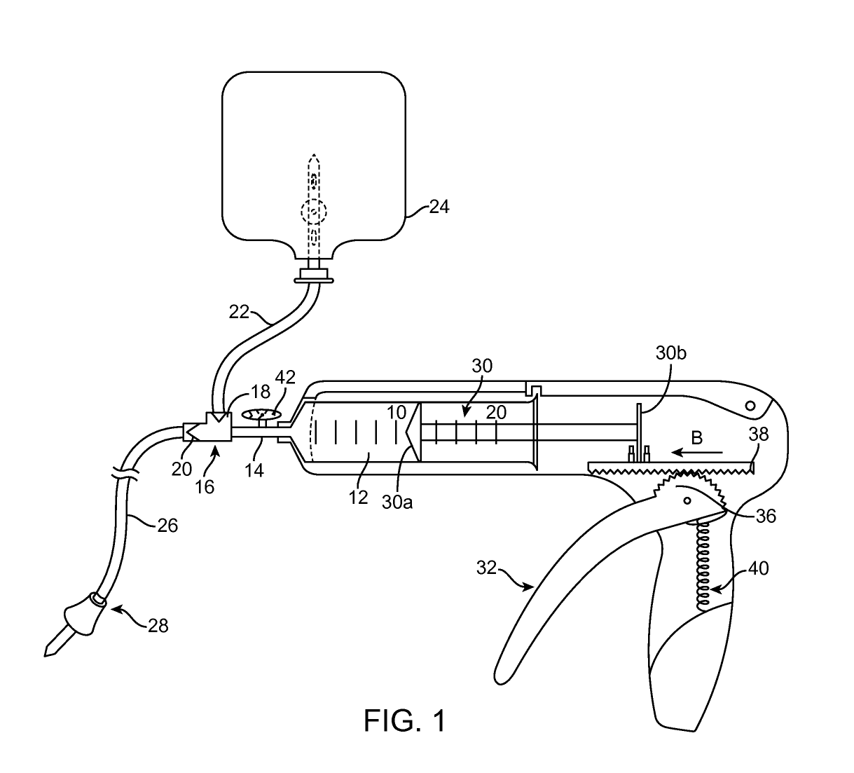

Referring to FIG. 1, fluid injection system 100 includes a housing 10 and a fluid chamber 12 supported by the housing. In the illustrated embodiment, the fluid chamber 12 comprises the barrel of a syringe such as a 20 ml syringe. The housing 10 may be configured to be reusable and designed such that a fresh, sterile, syringe may be removably coupled to the housing 10 prior to each use. In other embodiments, the housing 10 has an integrated chamber 12 and is disposable or sterilized between uses.

A valve housing 16 is fluidly coupled to the fluid chamber 12. In the FIG. 1 embodiment, the valve housing includes a proximal port/opening in communication with or coupled to a tubular port 14. Tubular port 14 is fluidly coupled to a distal portion of the chamber 12. A pressure measurement device 42 such as a manometer is positioned to measure pressure within the tubular port 14.

Valve housing 16 includes first and second one-way valves 18, 20. It should be noted, however, that in alternate embodiments the first and second one-way valves 18, 20 are not within a common housing.

A first conduit 22, which may be a length and caliber of tubing suitable for facilitating fluid passage at high flow rates, is coupled between the first valve 18 and a fluid reservoir 24 (e.g. saline or blood, and/or medication). A second conduit 26 is coupled between second valve 20 and an intraosseous port/needle 28 positioned with its distal end within bone marrow as described above. First valve 18 is oriented to allow one-way flow of fluid from reservoir 24 and conduit 22 into the tubular port 14 and chamber 12. The tubing 22, valve 18, tubular port 14, and distal tip of chamber 12 are of sufficiently large caliber and sufficiently short length to allow free and rapid flow of fluid into chamber 12 as spring mechanism 40 acts to retract plunger 30. Second valve 20 is oriented to allow one-way flow of fluid from chamber and tubular port 14 to conduit 26 and intraosseous port 28.

A plunger 30 includes a distal end 30a moveable within the chamber 12 between proximal and distal positions. Movement of the plunger in a distal direction expels fluid from the chamber 12 through the tubular port 14. The system includes an actuator that provides a mechanical advantage necessary to drive the plunger 30 and overcome the expected resistance to flow into the bone marrow. One example of an actuator, which drives the plunger using manual force, will be described with reference to FIG. 1. It should be understood, however, that various alternative actuators (including those using motors, pneumatics, or other sources of force to drive the plunger) might instead be used. The actuator is configured to generate fluid pressures adequate to overcome the resistance to flow into the bone marrow, and preferably to generate pressures (e.g. 300-450 mm/hg or higher) that will to achieve adequate flow rates into the IO space. While the optimal flow rate for a patient will depend on the size of the patient, the disclosed actuator allows average flow rates of at least 50 ml/min, and preferably 50-200 ml/min, to be achieved, far exceeding flow rates that can be achieved using conventional systems.

Referring again to FIG. 1, a handle on the housing 10 is engageable by a user to drive the plunger 30 distally. Handle includes a grip 32 pivotally coupled to the housing 10 at pivot 34. A pinion mechanism 36 on grip 32 is rotatable about pivot 34 when grip 32 is pivoted relative to the housing 10. A rack mechanism 38 includes gear teeth engaged with corresponding teeth on the pinion mechanism 36, and is slidable between proximal and distal positions. Movement of the rack mechanism 38 is mechanically coupled to the proximal end 30b of the plunger, such as through a direct or indirect connection between the rack 38 and proximal end 30b. Movement of the grip 32 in direction indicated by arrow A rotates pinion mechanism 36, which causes rack mechanism 38 to slide distally relative to the housing 10 as indicated by arrow B and to thus drive the plunger 30 distally.

The fluid injection system 100 preferably includes a mechanism for retracting the plunger 30 in a proximal direction upon release of the grip. In the FIG. 1 embodiment, a spring 40 biases the grip 32 such that when manual pressure against the grip 32 is released, the grip 32 pivots opposite to direction A, thus causing rack mechanism 38 to withdraw the plunger 30 proximally. In other embodiments, alternative mechanisms may be used to bias the plunger 30 in a proximal position.

### Method

Use of the fluid injection system 100 will next be described. First, a syringe is mounted to the housing 10 and positioned with its outflow port in fluid communication with tubular port 14 and with the proximal portion 30b of plunger 30 coupled to the actuator (e.g. the rack mechanism 38). Prior to coupling a fluid reservoir 24 to the system, the user squeezes the grip 32 in direction A, driving the rack mechanism 38 and thus the plunger 30 distally. With the plunger 30 maintained in the distal position (a latch may provided to engage the plunger in this position when needed), fluid reservoir 24 is fluidly coupled to first valve 18 via tubing 22. The user releases the grip 32, allowing the spring 40 to pivot the grip 32 to its resting position and to thus cause rack mechanism 38 to withdraw the plunger 30 to a proximal position. Retraction of the plunger 30 draws fluid from the fluid reservoir 24 through tubing 22 and valve 18 and into chamber 12 through tubular port 14. Each of these components is of sufficiently large bore (i.e. larger than more standard 3 mm diameter tubing and valves used in IO systems) to allow rapid and low-resistance flow into chamber 12 so that the user may rapidly administer the subsequent dose of fluid.

An intraosseous port 28 is fluidly coupled to tubing 26 and second valve 20. The port 28 is positioned with its distal tip within bone marrow as described above. The user squeezes the grip 32 in direction A, driving the rack mechanism 38 and thus the plunger 30 distally. Distal movement of the plunger 30 drives fluid from the chamber 12 through valve 20, and tubing 26, and into the bone marrow through the intraosseous port 28. Once the chamber 12 has been emptied, the user releases the grip 32, allowing the chamber 12 to be rapidly refilled from the fluid reservoir 24 as described in the preceding paragraph. The process is repeated, with the user repeatedly squeezing and releasing the grip to alternating deliver fluid and refill the chamber 12, until the appropriate volume of fluid has been administered.

The pressure measurement device 42 allows the user to monitor infusion pressures throughout fluid delivery, so as to avoid infusion pressures that can disrupt intraosseous needle placement. In some embodiments, the pressure measurement device 42 may include a visual or auditory indicator to alert the user when pressures exceed a threshold level so that the user may release the grip or maintain its position until such time as additional fluid can be safely delivered.

While certain embodiments have been described above, it should be understood that these embodiments are presented by way of example, and not limitation. It will be apparent to persons skilled in the relevant art that various changes in form and detail may be made therein without departing from the spirit and scope of the invention. This is especially true in light of technology and terms within the relevant art(s) that may be later developed.

What is claimed is:

1. A system for intraosseous delivery of fluids, the system comprising:
a housing configured to receive a chamber having a plunger moveable between distal and proximal positions within the chamber, the housing including an actuator moveable between first and second positions to move the plunger between the distal and proximal positions;
a first one-way valve fluidly couplable to a fluid reservoir, and
a second one-way valve fluidly couplable to an intraosseous port;
a tubular port fluidly couplable to the chamber,
wherein the valves are oriented such that
movement of the actuator from the first to the second position moves the plunger from the distal to the proximal position, drawing fluid from the fluid reservoir through the first valve and port and into the chamber; and
movement of the actuator from the second to the first position moves the plunger from the proximal to the distal position, driving fluid from the chamber through the port and second valve and through the intraosseous port.

2. The system of claim 1, further including a valve housing including the first and second one-way valves, wherein the tubular port extends from the valve housing.

3. The system of claim 1, further including a pressure measurement device positioned to measure pressure indicative of the pressure of fluid flowing from the intraosseous port.

4. The system of claim 2, wherein the pressure measurement device measures pressure within the tubular port.

5. The system of claim 1, wherein the actuator is biased in the second position.

6. The system of claim 1, wherein the actuator includes a grip pivotable relative to the housing and a member linearly moveable in response to pivoting of the grip.

7. The system of claim 6, wherein the grip includes a pinion mechanism and the member comprises a rack mechanism engaged with the pinion mechanism.

8. The system of claim 1, wherein movement of the actuator from the second to the first position causes the plunger to drive fluid into bone marrow at a pressure of at least 250 mmhg.

9. The system of claim 1, wherein movement of the actuator from the second to the first position cause the plunger to drive fluid into bone marrow at a pressure of at least 300 mmhg.

10. The system of claim 5, further including a spring biasing the actuator in the second position.

11. A system for intraosseous delivery of fluids, the system comprising:
a housing including a chamber and a plunger moveable between distal and proximal positions within the chamber, the housing including an actuator moveable between first and second positions to move the plunger between the distal and proximal positions;
a first one-way valve fluidly couplable to a fluid reservoir, and
a second one-way valve fluidly couplable to an intraosseous port;
a tubular port fluidly couplable to the chamber,
wherein the valves of the valve housing are oriented such that
movement of the actuator from the first to the second position moves the plunger from the distal to the proximal position, drawing fluid from the fluid reservoir through the first valve and port and into the chamber; and
movement of the actuator from the second to the first position moves the plunger from the proximal to the distal position, driving fluid from the chamber through the port and second valve and through the intraosseous port.

12. A system for intraosseous delivery of fluids, the system comprising:
a housing;
a chamber engageable with the housing and a plunger moveable between distal and proximal positions within the chamber, the housing including an actuator moveable between first and second positions to move the plunger between the distal and proximal positions;
a fluid reservoir;
an intraosseous port;
a first one-way valve fluidly coupled to the fluid reservoir, and
a second one-way valve fluidly coupled to the intraosseous port;
a tubular port fluidly coupled to the chamber,
wherein the valves of the valve housing are oriented such that
movement of the actuator from the first to the second position moves the plunger from the distal to the proximal position, drawing fluid from the fluid reservoir through the first valve and port and into the chamber; and
movement of the actuator from the second to the first position moves the plunger from the proximal to the distal position, driving fluid from the chamber through the port and second valve and through the intraosseous port.

13. A method for intraosseous delivery of fluids, comprising:
providing a first valve and a second valve;
fluidly coupling a fluid reservoir to the first valve;
fluidly coupling the first and second valves to a chamber having a plunger;
moving the plunger from a distal position to a proximal position within the chamber, thereby drawing fluid from the reservoir through the first valve into the chamber;
positioning a tip of an intraosseous port within bone marrow of a patient and fluidly coupling the intraosseous port to the second valve;
operating an actuator to move the plunger from the proximal position to the distal position, thereby driving fluid from the chamber through the second valve and out the intraosseous port into the bone marrow.

14. The method of claim 13, wherein the method provides a valve housing having the first and second valves, wherein the valve housing is fluidly coupled to the chamber.

15. The method of claim 13, further including, after driving fluid into the bone marrow, re-filling the chamber by moving the plunger from the distal position to the proximal position to draw fluid from the reservoir through the first valve into the chamber.

16. The method of claim 15 wherein the actuator is biased to position the plunger in the proximal position, wherein operating the actuator to move the plunger to the distal position includes applying manual force to the actuator against the bias, and wherein re-filling the chamber includes releasing the manual force from the actuator to allow the plunger to return to the proximal position.

17. The method of claim 13, wherein the chamber is carried by a device housing, and wherein operating the actuator includes pivoting a grip relative to the device housing.

18. The method of claim 17 wherein the grip is biased to position the plunger in the proximal position, wherein operating the actuator to move the plunger to the distal position includes applying manual force to the grip against the bias, and wherein re-filling the chamber includes releasing the manual force from the grip actuator to allow the plunger to return to the proximal position.

19. The method of claim 17, further including the step of coupling the chamber to the device housing and operatively engaging the plunger and actuator.

20. The method of claim 13, further including monitoring pressure of fluid flowing to the bone marrow.

21. The method of claim 16, further including within the actuator sufficient mechanical advantage necessary to overcome expected resistance to fluid flow during plunger movement to the distal position

22. The method of claim 16, further including large bore tubing and valve housing of sufficient caliber to allow low-resistance and rapid flow of fluid into the chamber during movement of plunger to proximal position

23. The method of claim 13, wherein the plunger drives the fluid at a pressure of at least 250 mmhg to overcome resistance to flow of fluid into the bone marrow.

24. The method of claim 13 wherein the plunger drives the fluid into the bone marrow at a rate of 40 ml/min or higher.

25. The method of claim 13 wherein the plunger drives the fluid into the bone marrow at a rate of 50 ml/min or higher.
